(19) United States
(12) Patent Application Publication
(10) Pub. No.: US 2011/0027907 A1
(43) Pub. Date: Feb. 3, 2011

(54) SSL7 MUTANTS AND USES THEREFOR

(75) Inventors: John David Fraser, Auckland (NZ); Bruce David Wines, Victoria (AU)

Correspondence Address: OCCHIUTI ROHLICEK & TSAO, LLP, 10 FAWCETT STREET, CAMBRIDGE, MA, 02138, US

(73) Assignee: Auckland Uniservice Limited, Auckland (NZ)

(21) Appl. No.: 12/445,675

(22) PCT Filed: Oct. 18, 2007
(86) PCT No.: PCT/NZ2007/000315

§ 371 Date: Oct. 21, 2010

(30) Foreign Application Priority Data
Oct. 20, 2006 (AU) 2006905858

(51) Int. Cl. G01N 33/68; C07K 14/31; C07H 21/00; C07K 1/22; C07K 1/14

(52) U.S. Cl. 436/501; 530/350; 536/23.7; 530/395; 530/412

(57) ABSTRACT

The invention relates to SSL7 mutants which have no, or at least reduced, ability to bind to IgA. The mutants have significant application in the purification or isolation of C5 from samples and in the identification or detection, including quantitation, of C5 in samples. Use of the mutants has the benefit of minimising or preventing simultaneous isolation and/or detection of IgA in a sample, simplifying and improving methods relying on wild type SSL7.

### FIELD

The present invention relates to mutants of SSL7 (also known as SET1) proteins and methods of use thereof. More particularly the invention relates to mutants of SSL7 which selectively bind serum complement factor C5 and their use in procedures for identification and/or isolation of C5.

### BACKGROUND

SSL7 is a staphylococcal superantigen-like protein (SSL) (otherwise referred to as staphylococcal exotoxin-like proteins (SETs)) expressed in the gram-positive bacterium Staphylococcus aureus. The SSLs, encoded by genes clustered within the staphylococcal pathogenicity island SaPIn2 are superantigen homologues. The function or role of SETs is unknown but they do not possess any superantigen activity despite ancestral relatedness. However, the presence of the SSL genes on SaPIn2 may indicate that they are part of the bacterial defense armamentarium (11) (8) (12). Notably an set15⁻ mutant of S. aureus displayed a 30-fold reduction in bacterial persistence in a murine kidney abscess infection model. Twenty-six members of the SSL family have been identified (8) (3) (10), although several appear to be allelic variants; for example SET1 (SSL7) from strain NCTC6571 (8), SET11 from N315 and Mu50 (3), and SET22 from MW2 (10) are probably the same protein.

Complement C5 is the central component in the terminal stage of the classical, alternative, and lectin mediated complement pathways. Complement C5 is ˜189 kD and is synthesised as an intracellular single-chain precursor that is secreted as a two-chain glycoprotein consisting of a 75 kD N-terminal C5β fragment disulfide linked to a 115 kD C-terminal C5α fragment ((23, 24)). The surface bound C5 convertases generated from either the classical, alternative or lectin pathway; cleave soluble C5 to generate two active fragments C5a and C5b. The potent anaphylatoxin C5a is a 74-residue N-terminus fragment cleaved from C5α by C5 convertase. C5a binds a G-protein coupled receptor C5aR on the surface of myeloid cells to stimulate a range of pro-inflammatory and chemotactic actions such as oxidative burst, phagocytosis and leukocyte recruitment which all contribute to the defense against organisms such as S. aureus (25). The C5b fragment initiates assembly of the terminal complement components into the membrane attack complex (MAC) that forms a water permeable membrane channel leading to cell lysis.

Recombinant SSL7 expressed in E. coli has been shown to independently and selectively bind to IgA and to serum complement factor C5 from a number of different species (WO2005/090381). WO2005/090381 describes methods for the identification and/or isolation of IgA and C5 which involve 1) bringing SSL7 into contact with a sample to allow it to bind to IgA and/or C5 to form a complex, and then either 2) detecting the bound SSL7, or 3) separating the complex, and releasing the IgA and/or C5 from the complex. While these methods provide a useful means of identifying and isolating both IgA and C5 it may be considered to be complicated by the fact that both IgA and C5 can bind simultaneously to SSL7. This may cause difficulties where one wishes to readily identify, detect, quantify or isolate only C5 for example.

Other methods to purify C5 from human serum, for example, are generally complex and rely on multiple chromatographic steps such as ion exchange and size exclusion chromatography. In addition they may often result in low yields of final product.

Accordingly, there may be considered a need to provide an alternative or improved method of isolating and identifying C5.

Bibliographic details of the publications referred to herein are collected at the end of the description.

### OBJECT

It is an object of the present invention to provide novel mutants of SSL7 and methods of use thereof.

### STATEMENT OF INVENTION

In a first aspect of the invention there is provided an SSL7 mutant having the ability to bind C5 but no or reduced ability to bind IgA.

Preferably the SSL7 mutant comprises an SSL7, allelic variant or functional equivalent thereof including one or more mutation in the IgA binding region. Preferably, the IgA binding region has been deleted.

Preferably the SSL7 mutant comprises an SSL7, allelic variant or functional equivalent thereof including a mutation at one or more of the following amino acid sites: 11, 14, 18, 36, 37, 38, 39, 55, 78, 79, 80, 81, 82, 83, 85, 87, 89 and 179.

Preferably the SSL7 mutant comprises an SSL7, allelic variant or functional equivalent thereof including a mutation at one or more of the following amino acid sites: Tyr11, Lys14, Arg18, Asn36, Tyr37, Asn38, Gly39, Phe55, Glu78, Leu79, Ile80, Asp81, Pro82, Asn83, Arg85, Ser87, Val89 and Phe179.

Alternatively, the SSL7 mutant comprises an SSL7, allelic variant or functional equivalent thereof including a mutation at one or more of the following amino acid sites: Leu10, Tyr11, Asp12, Lys14, Asp15, Arg18, Glu35, Asn36, Tyr37, Asn38, Gly39, Ser40, Phe55, Leu57, Lys77, Glu78, Leu79, Ile80, Asp81, Pro82, Asn83, Arg85, Ser87, Val89 and Phe 179.

Alternatively, the SSL7 mutant comprises an SSL7, allelic variant or functional equivalent thereof including a mutation at one or more of the following amino acid sites: Glu35, Ser40, Asn41, Val42, Arg44, Gln50, Asn 51, His52, Gln53, Leu54, Leu56, Leu57, Lys61, Val76, Lys77, Gly84, Leu86, Ser87, Thr88, Gly90, Lys133, Lys176, and Met182.

Preferably the SSL7 mutant is chosen from an SSL7, allelic variant or functional equivalent thereof including a mutation at one or more of the following amino acid sites: 37, 38, 44, 79, 81, 82, 83, and 85.

Preferably the SSL7 mutant is chosen from an SSL7, allelic variant or functional equivalent thereof including one or more of the following mutations:

### Y37A

### N38T

### R44A

### L79A

### D81A

### P82A

### N83A

### R85A

Alternatively, the SSL7 mutant comprises a C-terminal fragment of SSL7. Preferably the mutant comprises the amino acid sequence:

SSETNTHLFVNKVYGGNLDASIDSFSINKEEVSLKELDFKIRQHLVKNYG
LYKGTTKYGKITINLKDGEKQEIDLGDKLQFERMGDVLNSKDINKIEVTL
KQI.

In another broad aspect, the invention provides nucleic acids encoding an SSL7 mutant as herein before described.

In another broad aspect of the present invention there is provided a method of isolating C5 present in a sample, the method comprising at least the steps of:

Bringing an SSL7 mutant having the ability to bind C5 but no or reduced ability to bind IgA in contact with the sample for a period sufficient to allow the SSL7 mutant to bind to C5 to form a complex;
Separating the complex; and
Releasing C5 from the complex.

In a preferred aspect of the present invention there is provided a method for isolating C5 from a sample, the method comprising at least the steps of:

Providing a matrix to which an SSL7 mutant having the ability to bind C5 but no or reduced ability to bind IgA is bound;
Providing a sample;
Bringing said matrix and said sample into contact for a period sufficient to allow the SSL7 mutant to bind to C5 present in the sample; and,
Releasing C5 from the matrix.

Preferably, the method further comprises the step of collecting the C5 released.

Preferably, the matrix is in the form of a column over which the sample is passed.

Preferably the method further comprises the step of washing contaminants present in the sample from the matrix prior to release of C5.

Preferably the matrix is Sepharose.

Preferably the sample is milk or colostrum. More preferably the sample is serum.

Preferably the method further comprises the step of determining the quantity of C5 present in the sample.

Preferably, C5 is released low pH buffer such as 50 mM acetate pH 3.5.

In another aspect the invention provides a method of detecting C5 in a sample, the method comprising at least the steps of:

Contacting a sample with an SSL7 mutant having the ability to bind C5 but no or reduced ability to bind IgA for a period sufficient to allow the SSL7 mutant to bind to C5; and,
Detecting bound SSL7.

Preferably, the method further includes the step of determining or quantifying the level of bound SSL7.

Preferably, the method is conducted for the purpose of diagnosing C5 abnormalities in a subject.

Preferably the subject is a mammal, more preferably a human.

In another aspect of the invention there is provided a method of removing C5 from a sample, the method comprising at least the steps of:

Bringing an SSL7 mutant having the ability to bind C5 but no or reduced ability to bind IgA in contact with the sample for a period sufficient to allow the SSL7 mutant to bind to C5 to form a complex;
Separating the complex from the sample.

In another aspect, the invention provides a kit for the detection of C5 in a sample, the kit comprising at least an SSL7 mutant having the ability to bind C5 but no or reduced ability to bind IgA.

In a further aspect, the invention provides a kit for the isolation of C5 from a sample, the kit comprising at least an SSL7 mutant having the ability to bind C5 but no or reduced ability to bind IgA.

In another aspect, the invention provides a kit for the removal of C5 from a sample, the kit comprising at least an SSL7 mutant having the ability to bind C5 but no or reduced ability to bind IgA.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### FIGURES

These and other aspects of the present invention, which should be considered in all its novel aspects, will become apparent from the following description, which is given by way of example only, with reference to the accompanying figures, in which:

FIG. 1. Illustrates the crystal structure of the 2:1 complex of two SSL7 molecules bound to recombinant IgA Fc at 3.2 Å resolution. FIG. 1A is a front image and B is an “edge on” view of the of the 90° rotated complex. Molecules are depicted as ribbons with one SSL7 molecule (chain C) coloured blue at the N-terminus transitioning through grey to red at the C-terminus. The N-terminus and ajoining OB-domain (blue-grey coloured) of the SSL7 contributes all but one residue (Phe179) to the interaction with the IgA Fc. The two chains of the IgA Fc are depicted as orange and light blue ribbons. The buried interaction interface on the A chain of the Fc is shown as an aqua coloured surface. The major site of interaction of each SSL7 molecule with an IgA chain is centred on Leu441 at the Cα2/Cα3 interface but is extensive continuing down to the C-terminus of the Fc.

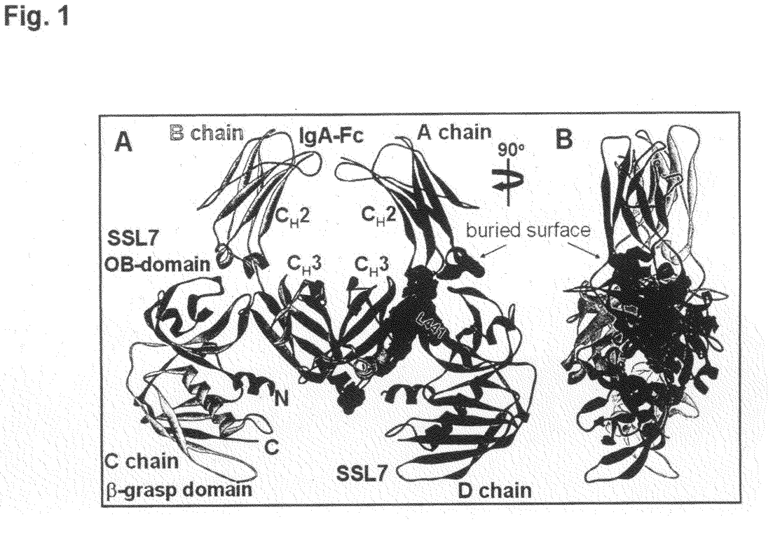

FIG. 2. Illustrates the bound interfaces between SSL7 (chain D) and IgA Fc (chain A, only) in the complex. The molecules are shown as a Cα trace with the SSL7 coloured blue at the N-terminus to red at the C-terminus and the A chain of IgA aqua and the B chain orange. Residues with a ≦4 Å separation of non-hydrogen atoms between the interacting chains are depicted in stick style. The SSL7 interface residues are coloured red or orange and the IgA residues coloured grey or magenta. Key features are the deep penetration of the OB fold b4/b5 loop, notably Pro82 at its end, into the Cα2/Cα3 interface. There is an inter-digitation of loops between the two molecules as the Cα3 FG loop residue Leu411 extends into a hydrophobic slot formed by SSL7 residues Phe55, Leu79-Asn81, Val89 and Phe179. Hydrogen bonding between the two interfaces potentially utilizes Tyr37, Asn38 and Asn83 of the SSL7 interface and the residues Leu257, Glu437 and Leu258 of the IgA interface. The interactions of the SSL7 (D) with the second chain of the Fc (B) have been omitted for clarity but these do interact (FIG. 3C). Similar analysis of the other SSL7 (chain B) indicates additional H bonds may be formed by residues Lys14, Lys14 and Asn38 with the IgA residues Asp449, Arg450 and Glu439 respectively.

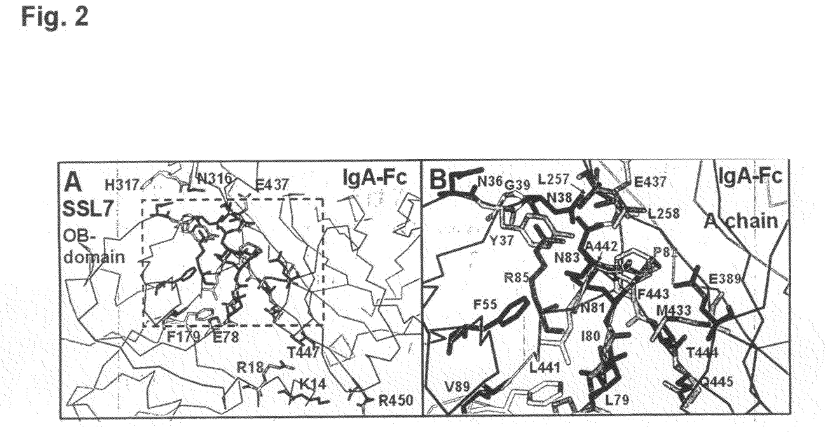

FIG. 3. Illustrates the SSL7 (chain D) and IgA interaction rendered as molecular surfaces. The surfaces of non-contact residues (>4 Å) are coloured grey and the SSL7 residues which contact the IgA Fc chain A are coloured red, those that contact the IgA Fc B chain are coloured brown and the corresponding contact residues on the IgA Fc A and B chains are coloured aqua and orange respectively. FIG. 3B shows the two interfaces of the bound complex. FIG. 3A shows the SSL7 molecule alone rotated 90° to show the contact surface end on. FIG. 3C shows the IgA Fc alone, rotated 90° to show the complementary contacting surface.

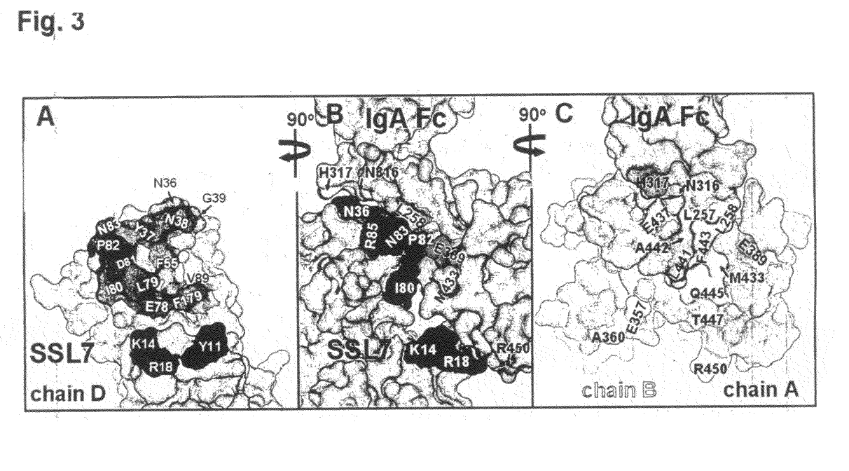

FIG. 4. Illustrates a pairwise depiction of contacts between polypeptide chains in the SSL7:IgA Fc complex. The aminoacid sequence of the chains (starting at SSL7 residue 10) is shown with contacts between the chains having a ≦4 Å separation of non-hydrogen atoms indicated by “*” and connected by a line to the interacting residue in the complementary interface.

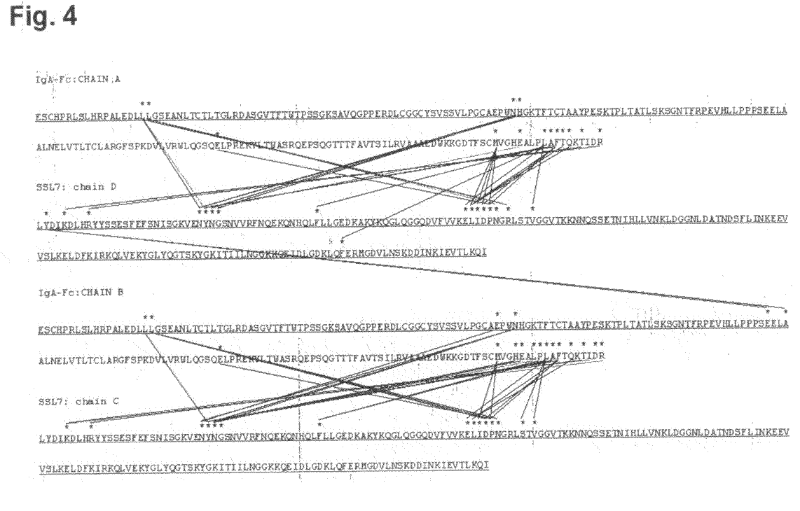

FIG. 5. Illustrates SSL7 and FcaRI binding activities of wild type and Cat mutant IgA proteins. WT, Leu256Ala, Leu257Ala, Leu258Ala, Asn316Ala and His317Ala mutant IgA fusion proteins were expressed transiently in CHOP cells and analyzed for biotinylated rSSL7 binding (A), apparent surface expression with FITC label anti-IgA (B), and Fc_RI-Ig binding (C).

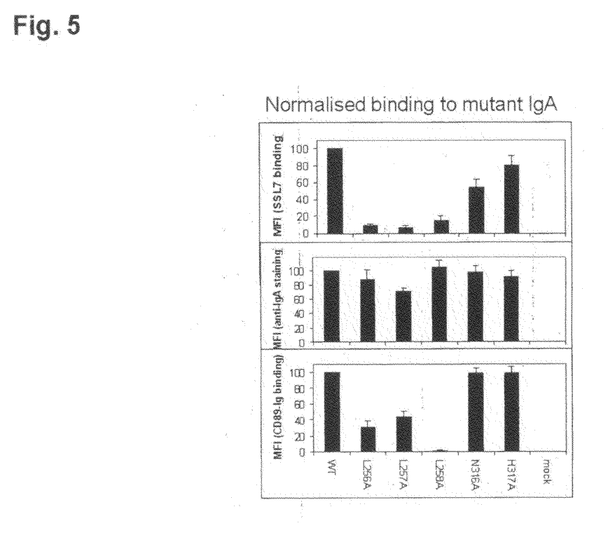

FIG. 6. Illustrates the relative contribution by amino acids from each SSL7 molecule bound to IgA Fc to the binding interface. The crystal structure coordinates were submitted to the protein interaction server (http://www.biochem.ucl.ac.uk/bsm/PP/server/index.html) and the results provided in tabular form with those residues listed that were found by calculation to contribute to the dimer interface. The results are presented for each chain of SSL7 interacting with the two separate chains of IgA Fc. Results are expressed as both absolute surface area (ASA) results and as a percentage of the total surface (% Interface) bounded by the protein-protein interactions.

FIG. 7. Illustrates an alignment of various SSL7 amino acid sequences as published in GenBank. IgA binding residues are underlined. SSL7 (GL1) protein was used in the co-crystallisation of SSL7 with IgA. The following sequence identification numbers, have been allocated to the amino acid sequences provided in FIG. 7: SSL7 (4427) is SEQ ID No: 1, GL10 is SEQ ID NO: 21, MW2 is SEQ ID NO: 22, GL1 is SEQ ID NO: 23, N315 is SEQ ID NO: 24, Mu50 is SEQ ID NO: 25, NCTC8325 is SEQ ID NO: 26 AND consensus sequence is SEQ ID NO: 27.

FIG. 8. Illustrates the proteins purified on a C-terminal SSL7 protein bound to Sepharose from human serum. Lane 1—protein standards; lane 2—serum proteins not bound; lane 3—bound proteins eluted with 1M MgCl2; lane 4—bound proteins eluted with 0.1M glycine pH3.5; lane 5—bound proteins eluted with 0.1M glycine pH3.0; lane 6—bound proteins eluted with 0.1M glycine pH2.9. The bands at 110 kD and 70 kD in lanes 5 and 6 are complement C5.

FIG. 9. Illustrates the effects of mutations D117A and E170A on the ability of SSL7 to inhibit complement mediated haemolysis. Recombinant SSL7 or SSL7 mutant proteins were added in a dose dependent fashion to human serum and human red blood cells. The degree of red cell lysis was measured by the release of haemoglobin at 460 nm.

### PREFERRED EMBODIMENT(S)

The following is a description of the present invention, including preferred embodiments thereof, given in general terms. The invention is further elucidated from the disclosure given under the section “Examples” which provides experimental data supporting the invention and specific examples thereof.

SSL7 binds independently to serum complement factor C5 and to IgA allowing for simultaneous binding to both molecules. Using co-crystal structure analysis and mutation studies the inventors have elucidated the IgA binding site on SSL7 and have identified amino acid residues which they believe are key for IgA binding to SSL7. The inventors have also generated SSL7 mutants which have no, or at least reduced, ability to bind to IgA. These mutants have significant application in the purification or isolation of C5 from samples and in the identification or detection, including quantitation, of C5 in samples. Use of the mutants has the benefit of minimising or preventing simultaneous isolation and/or detection of IgA in a sample, simplifying and improving methods relying on wild type SSL7.

The purification of complement C5 from samples, such as serum, has a number of uses. For example, it could aid in the study of complement mediated immune disorders and the study of the mechanisms of inflammation. It may also be of use in the production C5 protein as a research reagent on a commercial basis. In addition, detection of complement C5 levels in serum may be of use in identifying or diagnosing defects in complement activation related to reduced levels of C5 in patient sera.

It should be appreciated that reference to isolation, removal, detection or quantifying the level of C5, may include isolation, removal, detection or quantifying the level of a subunit or monomer of C5, or fragments of C5 where appropriate.

Further, it should be appreciated that reference to C5 should be taken to include reference to any alternative forms of this molecule (for example allelic variants, fusion proteins, modified versions of C5 from different species) which are capable of binding to the SSL7 mutants of the invention.

As used herein “SSL7”, or “wild type SSL7”, refers to a protein having an amino acid sequence exemplified by one or more of AAF05587 (SET1_NCTC8325), BAB41615 (SET11_N315), NP_{—}370950.1 (SET11_Mu50), NP_{—}645205.1 (SET22_MW2), SEQ ID NO: 6 of WO2005/090381 (SET1 GL10 S. aureus Greenlane), SEQ ID NO: 7 of WO2005/090381 (SET1 GL1 S. aureus Greenlane) and SSL7 (4427) as described herein after), or allelic variants or functional equivalents of any one of the foregoing.

As will be appreciated by persons of skill in the art to which the invention relates the sequences of any known proteins or nucleic acids mentioned herein may be found on the NCBI database using the relevant accession numbers listed; for example AAF05587.

Allelic variants or functional equivalents of SSL7 include peptides or full length proteins having the ability to bind C5 and IgA, preferably C5 and IgA from human serum. The allelic variants or functional equivalents will typically have at least approximately 70% amino acid sequence similarity to an SSL7 exemplified above. Alternatively they will have at least approximately 80% amino acid sequence similarity, at least 85% amino acid similarity, at least approximately 90% sequence similarity, or at least approximately 95% similarity. The phrase “the ability to bind IgA and C5” should not be taken to imply a specific level of binding or affinity between the molecules or that they will have equal affinity for IgA and C5. Preferably the allelic variant or functional equivalent will have a dissociation constant towards C5 that is at least 1 nanomolar and more preferably greater than 1 micromolar.

An SSL7 protein may be from any species of animal.

Reference to SSL7 proteins and the exemplary sequences provided herein should be taken to include reference to mature SSL7 polypeptides excluding any signal or leader peptide sequences or other sequences not present in the mature protein but which may be represented on public databases for example. Persons of general skill in the art to which the invention relates will readily appreciate such mature proteins.

Nucleic acids encoding SSL7 proteins will be appreciated having regard to the amino acid sequence information herein and the known degeneracy in the genetic code. However, exemplary nucleic acids include AF188835 (SET1, NCTC6571), BAB41615.1 (SET11_N315), NP_{—}370950.1 (SET11_Mu50), NC_{—}003923.1 (SET22_MW2), SEQ ID NO: 12 of WO2005/090381 (SET1 GL10 S. aureus Greenlane), SEQ ID NO: 13 of WO2005/090381 (SET1 GL1 S. aureus Greenlane), and SSL7 (4427) as described hereinafter.

“SSL7 mutants” of the invention have the ability to bind C5 but have reduced or no ability to bind IgA due to disruption of the IgA binding region compared to wild type SSL7.

As used herein “reduced ability” to bind IgA means any binding that is higher in dissociation constant (K_{D}) than the parent molecule as measured quantitatively by biosensor analysis between soluble IgA and SSL7. More preferably the mutant SSL7 has a binding that is more than 5-fold higher in dissociation constant (K_{D}) than the parent molecule as measured quantitatively by biosensor analysis between soluble IgA and SSL7.

Disruption of the IgA binding region of SSL7 may be achieved by altering or mutating individual or multiple amino acids that contribute to binding to IgA or otherwise support IgA binding. This may be achieved by substitution of one or more relevant amino acid with an alternative amino acid, or deletion of one or more relevant amino acid or the entire region that contains the IgA binding site. Persons of ordinary skill in the art to which the invention relates may appreciate alternative means for disrupting the IgA binding region, having regard to the information contained herein.

In one embodiment the IgA binding region may be disrupted by incorporating in SSL7 a mutation at one or more of the following amino acid sites which participate in contacts with the IgA: residues: 11, 14, 18, 36-39, 55, 78-83, 85, 87, 89 and 179.

More specifically, the IgA binding region may be disrupted by incorporating in SSL7 a mutation at one or more of the following amino acid sites which participate in contacts with the IgA (≦4 Å distance of SSL7 non-hydrogen atoms from IgA non-hydrogen atoms): residues Tyr11, Lys14, Arg18, Asn36, Tyr37, Asn38, Gly39, Phe55, Glu78, Leu79, Ile80, Asp81, Pro82, Asn83, Arg85, Ser87, Val89 and Phe179.

Alternatively, the IgA binding region may be disrupted by incorporating in SSL7 a mutation at one or more of the following amino acid sites which contribute to the buried surface area of a interface with the IgA: residues Leu10, Tyr11, Asp12, Lys14, Asp15, Arg18, Glu35, Asn36, Tyr37, Asn38, Gly39, Ser40, Phe55, Leu57, Lys77, Glu78, Leu79, Ile80, Asp81, Pro82, Asn83, Arg85, Ser87, Val89 and Phe179.

Furthermore inspection of the SSL7 structure reveals residues which contact other SSL7 residues (≦4 Å separation of non-hydrogen atoms) that directly bind IgA (≦4 Å distance of SSL7 non-hydrogen atoms from IgA non-hydrogen atoms) and thereby may contribute to the IgA binding activity of SSL7 by supporting the structure of the ligand contacting residues. Hence, the IgA binding region may be disrupted by incorporating in SSL7 a mutation at one or more of the following amino acid sites which may contribute to the structure of the binding regions: residues Glu35, Ser40, Asn41, Val42, Arg44, Gln50, Asn 51, His52, Gln53, Leu54, Leu56, Leu57, Lys61, Val76, Lys77, Gly84, Leu86, Ser87, Thr88, Gly90, Lys133, Lys176, Met182.

In one embodiment of the invention the IgA binding region is disrupted by incorporating a mutation at one or more of the following amino acid sites: 37, 38, 44, 79, 81, 82, 83, 85.

In a related embodiment, one or more of the following amino acid substitutions are incorporated in SSL7:

### R44A

### Y37A

### N38T

### L79A

### D81A

### P82A

### N83A

### R85A

In another embodiment, the whole IgA binding region of SSL7 is deleted. In this embodiment the mutant comprises a C terminal fragment of SSL7. Preferably the C-terminal fragment comprises the amino acid sequence:

SSETNTHLFVNKVYGGNLDASIDSFSINKEEVSLKELDFKIRQHLVKNYG
LYKGTTKYGKITINLKDGEKQEIDLGDKLQFERMGDVLNSKDINKIEVTL
KQI (designated herein as SEQ ID No: 3).

The first serine in this sequence is found at position 99 of the SSL7 protein.

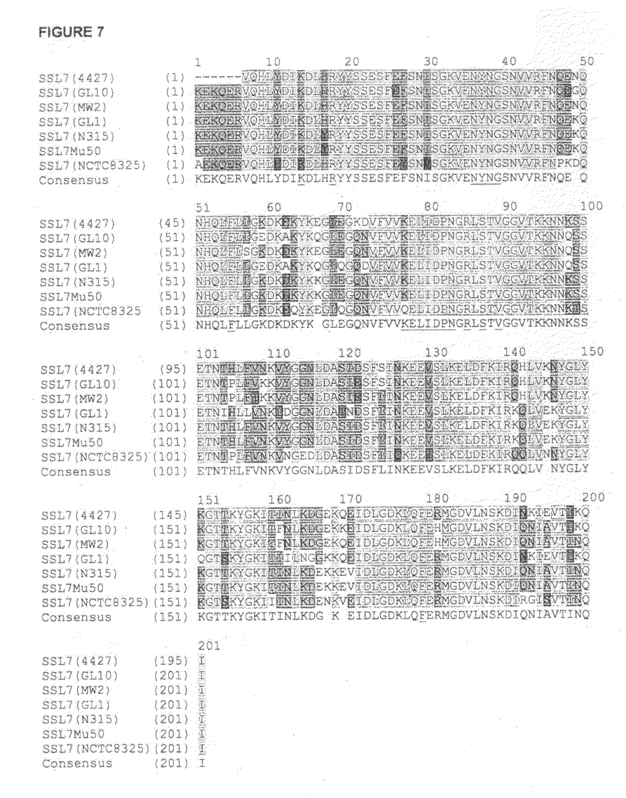

The amino acid positions mentioned herein are numbered from the start of the mature protein sequence of the SSL7 allele used by the inventors in studying the interaction between SSL7 and IgA. This allele was the GL1 allele isolated from a S. aureus strain from Greenlane Hospital (SEQ ID NO: 7 of WO2005/090381 (SET1 GL1 S. aureus Greenlane), and FIG. 7). The amino acids are numbered from the first K (Lysine) at the N-terminus as per FIG. 7. This corresponds to the first A of the commonly used reference sequence SSL7 (SET1 old nomenclature) obtained form the NCTC8325 (GenPep accession number AAF05587.1) also shown in FIG. 7.

The inventors contemplate the use of SSL7 mutants of the invention in the form of fusion proteins, provided the heterologous amino acid sequence does not substantially interfere with binding to C5. Similarly, SSL7 mutants of the invention may include non-naturally occurring or chemically modified amino acids where desirable.

Mutations in SSL7 may be introduced using known site directed mutagenesis techniques. For example, overlap PCR may be used as described in reference 36 and detailed further in the Examples section of this specification. Persons of ordinary skill in the art to which the invention relates may readily appreciate alternative mutagenesis techniques.

Once generated, an SSL7 mutant may be reproduced by any number of standard techniques known in the art, having regard to the amino acid and nucleic acid sequences identified herein before. By way of example, they may be produced recombinantly or produced by chemical synthesis.

Persons of general skill in the art to which the invention relates will readily appreciate nucleic acids of use in generating the mutants of the invention, as well as nucleic acids encoding the mutants, having regard to the amino acid sequences of various SSL7s and SSL7 mutants described herein as well as the known degeneracy of the genetic code. However, the following nucleic acid sequences of wild type SSL7s provide examples of relevant nucleic acids: AF188835 [SET 1 —S. aureus NCTC6571]; BAB41615.1 [SET 11—S. aureus N315]; NP_{—}370950.1 [SET 11 —S. aureus Mu50]; NC_{—}003923.1 [SET 22 —S. aureus MW2]; SEQ ID No: 12 [WO2005/090381—SET1—GL10 isolate]; SEQ ID No: 13 [WO2005/090381—SET1—GL1 isolate]; and SSL7 [4427] as described herein after.

In accordance with this aspect of the invention the invention also encompasses nucleic acids encoding the mutants of the invention, as well as nucleic acid vectors adapted for example to express or clone nucleic acids encoding the mutants, and host cells containing such vectors. Exemplary nucleic acid vectors and host cells are described herein after in the “Examples” section.

An ‘isolated’ nucleic acid as may be referred to herein, is one which has been identified and separated from at least one contaminant nucleic acid molecule with which it is associated in its natural state. Accordingly, it will be understood that isolated nucleic acids are in a form which differs from the form or setting in which they are found in nature. The term ‘isolated’ does not reflect the extent to which the nucleic acid molecule has been purified.

The efficacy of any mutant made in accordance with the present invention can be assessed by testing its ability to bind and/or inhibit C5, and by testing its ability to bind IgA. In relation to C5 binding recombinant mutant SSL7 is added to a haemolytic assay which measures the complement activity of human serum. Generally, washed human red blood cells are incubated for 30 minutes with 10% human serum from a patient with naturally occurring reactivity to the donor red cells. C5 mediated lysis is measured by the release of haemoglobin from the lysed red cells. The ability of SSL7 to bind and inhibit C5 is measured by introducing the SSL7 protein into the serum, prior to the addition of red cells. Inhibition by SSL7 is measured as a decrease in haemolysis.

In relation to IgA binding, the BIAcore biosensor assay described elsewhere herein is an example of an assay which may be used to identify appropriate mutants having at least reduced ability to bind IgA, preferably no ability to bind IgA.

One embodiment of the invention relates to a method for isolating C5 from a sample using an SSL7 mutant having the ability to bind C5 but no or reduced ability to bind IgA. Generally, the method comprises at least the steps of: bringing an SSL7 mutant having the ability to bind C5 but no or reduced ability to bind IgA into contact with the sample for a period sufficient to allow the SSL7 mutant to bind to C5 to form a complex; separating the complex; and, releasing C5 from the complex.

In a preferred form of this embodiment the method comprises at least the steps of: providing a matrix to which an SSL7 mutant having the ability to bind C5 but no or reduced ability to bind IgA is bound; providing a sample; bringing said matrix and said sample into contact for a period sufficient to allow the SSL7 mutant to bind to C5 present in the sample; and, releasing C5 from the matrix.

It should be understood that the terms “isolate”, or “isolating” and the like indicates that C5 has been separated from at least one contaminating compound. It should be appreciated that ‘isolated’ does not reflect the extent to which C5 has been purified.

In accordance with a preferred form of the invention, C5 is captured or isolated using affinity chromatography however a skilled person may readily recognise alternative techniques. Generally, an affinity column is prepared combining a SSL7 mutant, suitably immobilised on a support resin or matrix.

Any appropriate support resin as known in the art may be used. As it will be appreciated, choice of support resin may depend on the means by which the SSL7 mutant is to be immobilised on it. Preferable support resins include Sepharose such as Sepharose 4B, cyanogen bromide-activated (CNBr-activated) Sepharose, AH-Sepharose 4B and CH-Sepharose 4B, activated CH-Sepharose 4B, Epoxy-activated Sepharose 6B, activated Thiol-Sepharose 4B, Thiopropyl-Sepharose 6B, covalently cross-linked Sepharose (sepharose Cl), and other resins such as nickel chelate resins, cellulose, polyacrylamide, dextran. Such resins may be purchased for example from Pharmacia Biotech. However, a skilled person may produce a resin themselves using methodology standard in the art

While the inventors have found that it is not necessary to use spacer molecules it should be appreciated that where desirable, and where one is not present on a resin as it may be purchased or manufactured, a spacer molecule may be added to the resin. Such spacer molecule may, in certain circumstances, facilitate the attachment of the ligand (SSL7 mutant) to the resin, and also facilitate efficient chromatographic isolation of C5. Relevant spacer molecules will readily be appreciated by persons of ordinary skill in the art.

In addition, cross-linking of a support resin, or activation of resins may help facilitate chromatographic separation. Accordingly the invention encompasses this. While support resins which have been cross-linked and/or activated may be readily purchased (for example, Sepharose Cl or CNBr-activated Sepharose) skilled persons will readily appreciate methods for achieving such results themselves

It will be appreciated that SSL7 mutants may be chemically modified where necessary and to facilitate attachment to the support resin while not destroying its ability to bind C5.

Once the support resin is prepared and any modifications made to it and/or a SSL7 mutant, the SSL7 mutant may be immobilized on the support resin using standard methodology. By way of example, the protein and the resin may simply be mixed for a period of time (by way of example, 2 hours) to allow for attachment of the protein to the resin. Subsequently, any active groups which may remain on the resin may be blocked by mixing with a buffer such as Tris at pH 8.0 for a period of time (for example 2 hours). The protein-resin may then be washed in an appropriate buffer, such as PBS, then suspended in an appropriate buffer and stored. In a preferred from of the invention where a Sepharose resin is used, the protein-resin is stored 1:1 in a PBS/0.025% NaN₃ buffer at 4° C. until desired to be used.

The SSL7 mutant may be combined with the support resin in any desired ratio. In a preferred form of the invention using CNBr-activated Sepharose 4B, an SSL7 mutant is combined at 7 mg of protein/ml of wet gel Sepharose. This typically results in a concentration of approximately 5 mg protein/ml of Sepharose gel.

Once the affinity matrix or resin is prepared as mentioned herein before it may be formed into a column according to standard techniques readily known in the field. The column may then be washed with an appropriate buffer to prepare it for taking a sample. Such appropriate buffer includes for example PBS, or any other neutral pH buffer containing isotonic concentrations of NaCl. A sample may then be loaded onto the column and allowed to pass over the column. In this step, C5 present in a sample will adsorb to the column resin or matrix.

Once a sample has passed over the column it will generally be washed with an appropriate buffer to remove unbound or non-specific proteins or other compounds which may have been present in the original sample. Skilled persons will readily appreciate an appropriate buffer suitable for use. However, by way of example, a PBS/500 mM NaCl buffer may be advantageously used or alternatively 1M MgCl₂.

IgA may be eluted from the column using a solution that is buffered to pH 3.5. In a specific example, 10 column volumes of 50 mM acetatic acid pH 3.5 is used. However, it should be appreciated that this may be varied by substituting acetate with any other inert chemical such as glycine which buffers effectively in the range of pH 2.7-3.7.

For example, C5 may be eluted from the column using a solution that is buffered to pH 2.9-3.0. In a specific example, 5 column volumes of 100 mM glycine pH2.9 is used. C5 will generally be eluted into any buffer which is adapted to neutralize the low pH of the elution buffer. The inventors have found that 1M Tris pH 8.0 to 1/10^{th} the volume of eluate to be appropriate, but any similar buffer such as phosphate that raises the pH to neutral is suitable.

Following elution or release of C5 from the matrix or column it may be further purified via any number of standard techniques. For example, eluates may be dialysed, or run through an affinity column of the invention again.

It should be appreciated that a chromatographic column in accordance with the invention may be gravity fed, or fed using positive or negative pressure. For example, FPLC and HPLC are applicable to a method of the invention.

Skilled persons will readily appreciate how to implement an HPLC system in relation to the present invention having regard to the information herein and standard methodology documented in the art.

Persons of ordinary skill in the art to which the invention relates will readily appreciate how scale up of bench top columns may be achieved. For example, one may increase volume of the affinity column consistent with the volume of sample to be processed. Commercial scale may be dependent on ensuring that the amount of coupled SSL7 mutant saturates the amount of ligand to be bound. Alternatively, large amounts of sample may be processed by repeated processing through a smaller SSL7 mutant affinity column. This has the advantage of not requiring so much SSL7 mutant but does rely on the reusability of the SSL7 mutant for recycling. The inventors believe that the SSL7 mutants are very stable when used for purification of IgA and/or C5 and can be reused many times without loss of binding activity.

It should be appreciated that an affinity matrix can also be used in batch wise fashion where the solid matrix is added directly to the sample rather than passing the sample through a column. This offers simplicity, but may result in a less clean sample. Such techniques require a step to separate the matrix from the solution or sample. This is normally achieved by gravity sedimentation and decantation of the supernatant followed by washing, or separation of the affinity matrix by low pressure gravity or suction filtration.

The present invention has the advantage of providing a one step system for isolating C5. It should be appreciated that there may be instances where it is desirable to obtain a biological sample that is free from C5. The present invention will allow for substantial removal of C5 from a sample. The techniques described hereinbefore are suitable for achieving this end. It should be appreciated that where removal of C5 is the objective (as opposed to capture and purification of C5) it would not be necessary to release C5 from any SSL7 mutant to which it is bound.

In addition, the SSL7 mutants of the invention provide a means for detecting the presence, and quantifying the level, of C5 in a sample. This has diagnostic significance in determining complement competency (C5) and in detecting abnormalities in C5 (for example deficiencies, or increased expression) in a subject. Diagnostic methods involving detection and/or quantitation of C5 may find particular use in assessing the immune competence of an individual. For example, human C5 deficiencies could be readily detected by examining the ability of mutant SSL7s of the invention to selectively bind C5 present in the patients serum. The levels of C5 would be quantified against a standard curve of known C5 concentrations. Knowledge of immune competence may allow for more informed and individualised approaches to athletic training schedules, general nutrition, and medication regimes.

In accordance with the above, the invention also provides methods for detecting the presence, and/or quantifying the level of C5 in a sample. The method will generally comprise the steps: contacting a sample with a SSL7 mutant for a period sufficient to allow the SSL7 mutant to bind to C5; and, detecting the bound SSL7 mutant. The method preferably includes the further step of determining the level of bound SSL7 mutant. Such a method is applicable to any sample which may contain C5. It is applicable to samples from humans and other animals.

Persons of skill in the art to which the invention relates will appreciate means by which C5/SSL7 mutant can be detected and/or quantified. However, by way of example the SSL7 mutant may first be conjugated to peroxidase or alkaline phosphatase by chemical cross-linking using standard methods to produce a staining reagent. Samples can be added to ELISA plates and the SSL7 mutant can be added at a fixed concentration to bind to any C5 bound to the plastic plate surface. Following washing, the amount of SSL7 mutant can be quantified by measuring the amount of peroxidase or alkaline phosphatase bound using established colorimetric methods that result in the production of a coloured compound which can be measured in an ELISA plate reader. The levels of C5 in the sample can be determined by comparing results against a standard curve of a known sample of C5. An alternative example is to utilise a sandwich ELISA employing an anti-SSL7 mutant specific antibody. In this case the anti-SSL7 mutant antibody is conjugated to either peroxidase or alkaline phosphatase. After the SSL7 mutant has incubated with the sample on the ELISA plate and excess washed away, the anti-SSL7 mutant antibody linked to the enzyme is incubated and washed clean.

Appropriate “samples” from which C5 may be detected, quantified, captured or isolated in accordance with the invention include serum, bodily secretions or cell cultures utilised for recombinant production of C5. The sample may be of human, or other animal origin (for example rabbit) where the C5 from that species binds SSL7. Skilled persons may appreciate other samples to which the invention is applicable.

In other embodiments the invention provides a kit for use in one or more of the methods described herein. The kit will comprise at least an SSL7 mutant of the invention in a suitable container. The kit preferably also comprises, in separate containers, one or more buffers or washing solutions required to perform a method of the invention. The kit may also comprise appropriate affinity columns, matrices, or the like. In one embodiment, the kit comprises a column comprising a matrix to which an SSL7 mutant is already bound. Further, kits of the invention can also comprise instructions for the use and administration of the components of the kit.

Any containers suitable for storing compositions may be used in a kit of the invention. Suitable containers will be appreciated by persons skilled in the art.

### EXAMPLES

### Co-Crystal Structure of SSL7-IgA-Fc

### Methods and Materials

### Production of Recombinant IgA-Fc and SSL7.

Briefly a pENTR1A (Invitrogen Life Technologies, Melbourne Australia) construct containing a sequence encoding an anti-TNP chimeric IgA1 antibody with mouse VH from TIB142 (ATCC, Manassas, Va.) and a truncated human constant region from IMAGE cDNA clone 4701069 (Clontech laboratories and I.M.A.G.E. Consortium) with an in frame termination codon following the codon for Pro455 (pBAR378) was used as a template for PCR using accuprime Pfx (Invitrogen) and the oligonucleotide primers oBW328 TCCTGCCACCCCCGACTGTCAC (designated herein as SEQ ID No: 4) and oBW329 CTCTGACAGGATACCCGGAAGG (designated herein as SEQ ID No: 5). The PCR product was phosphorylated and ligated using standard molecular biology techniques and the construct encoding the mouse TIB142 leader sequence and IgA Fc region (Cys242 to Pro455; IgA1 myeloma Bur numbering) was sequenced using BigDye3.1 (ABI, Melbourne, Australia). This sequence was then inserted into pAPEX-3p-X-DEST (pBAR424) expression vector using LR clonase (Invitrogen). The vector pBAR424 consists of the Gateway RfA cassette inserted at the blunt ended XbaI site in the vector pAPEX-3p (30). The Fc was produced by transfection of HEK293EBNA cells using Lipofectamine 2000 and selection with 2 mg/ml puromycin (Sigma, Melbourne Australia). Purification from the supernatant used thioredoxin-SSL7 fusion protein coupled to cyanogen bromide Sepharose (GE, Melbourne, Australia) and elution with 50 mM glycine pH 11.5. The eluate was immediately neutralized. The production of recombinant SSL7 (GL1 S. aureus Greenlane) has been described previously (31).

### Transferrin Receptor (TfR)-IgA Fc Fusion Protein and IgA Fc Mutants.

The surface expression and assay of the SSL7 and FcaRI-Ig binding activities of the Fc region of IgA1, and mutants thereof, fused to the transmembrane region of the type II receptor transferrin was performed as previously (32).

### Crystallisation Conditions

Dialysed proteins were concentrated using a Macrosep 10K Omega concentrator (Pall Filtron). Conditions for crystallising SSL7 in complex with recombinant IgA-Fc were determined using the Hampton Research (Aliso Viejo, Calif., USA) Crystal Screen HT kit with the final conditions being IgA-Fc 9.7 mg/ml and SSL7 7.0 mg/ml mixed with an equal volume of 12% PEG 8000, 66 mM sodium cacodylate pH 6.5, 130 mM calcium acetate. The crystals used for data collection had the space group P2(1)2(1)2(1) and the unit cell dimensions; a=71.306 b=109.263 c=170.863.

### Results

Mutagenesis of the cα2 AB Loop in IgA Fc.

SSL7 inhibits the function of the IgA Fc receptor, FcαRI (CD89), as both SSL7 and FcαRI bind to the Cα2/Cα3 inter-domain region of the Fc. This interaction was investigated by making mutants of IgA, namely L256A, L257A, L258A, N316A and H317A, in the cα2 AB loop. The L256A and L257A mutations were the most deleterious reducing SSL7 binding 11.5-fold and 15.4-fold respectively. Since these changes are alterations in the length of the aliphatic side chain the complementarity of the interface between SSL7 and the IgA Fc is presumably affected. It is noteworthy that the L256A mutation adversely affects SSL7 binding activity of the IgA although this residue is not a contact residue in the binding interface and is a minor contributor (<2%) to the buried surface area. Thus mutation of Leu256 must affect the presentation of other residues in the Cα2 AB loop such as Leu257 and Leu258 and so indirectly affectingly SSL7 binding. The mutation L258A reduced SSL7 binding 6.5-fold and the N316A mutation had a lesser effect reducing binding 1.8-fold, while the effect of the H317A mutation was negligible (1.2 fold). Next the activities of these Fc mutants in FcαRI binding were examined. In contrast to their SSL7 binding activities the L256A and L257A Fc mutations resulted in a modest reduction, 3.3-fold and 2.3-fold, in FcαRI binding, while the L258A mutant had >100-fold reduction in FcαRI binding activity. Thus although these two proteins bind some IgA residues in common at the cα2/cα3 interface there are marked differences in the contributions that these residues make to the binding interaction. The mutagenesis data also indicates the SSL7 has a different footprint on the Fc to that of FcαRI, as evidenced by the lack of effect of the N316A and H317A mutations on FcαRI binding while there was a modest effect of the N316A mutation on SSL7 binding.

The observed SSL7 and FcαRI binding activities of the IgA Fc mutants L256A, L258A, N316A and H317A were not due to altered surface expression of these Fcs as these showed staining (MFI) with anti-IgA polyclonal antiserum in FACS analysis comparable to that of the WT (87-103%). Surface staining of the L257A mutant was reduced 1.4-fold (70%) of that of WT which was still considerably less than the decrease in SSL7 and FcαRI binding activities of 15.4-fold and 2.3-fold respectively.

The 3.24 Crystallographic Structure of SSL7 in Complex with IgA-Fc.

SSL7 is a superantigen related protein with a modular architecture comprising an N terminal OB fold and a C terminal β grasp domain. Two SSL7 molecules bound to a single IgA-Fc were found in the crystallographic unit cell with pseudo-two fold symmetry. Each SSL7 molecule essentially binds the same site at the cα2/cα3 interface of each chain of the Fc and >95% of the interacting surface is contributed by the OB-fold of the SSL7 molecules. There is asymmetry in the complex and some minor differences between the interactions of the two SSL7 molecules with the IgA which is most pronounced in the different interactions of the N-termini (residues 10 to 20) of the two SSL7 molecules. The interaction of this N-terminal region is minor in comparison with the other contacts with the IgA Fc. As such the alternate forms of the interaction of the N-termini of each of the SSL7 molecules in the complex may be fixed by crystal packing.

### Analysis of the SSL7:IgA Fc Interface.

The protein interface, a ≦4 Å separation of non-hydrogen atoms of the interacting chains, was analyzed using the program iMolTalk Structural Bioinformatics Toolkit (version: 3.1; available on the iMolTalk—the interactive Structure Analysis Server http://i.moltalk.org/) (33, 34). The SSL7 D chain (residues; 14, 18, 36-39, 55, 78-83, 85, 89, 179) were identified as contacting the IgA Fc A chain (residues; 257-258, 316-317, 389, 433, 437, 441-445, 447, 450) and an addition minor contact is made between SSL7 (D chain residue Tyr11) and the IgA Fc chain B (residues; 357,360). The contacts for the second SSL7 molecule (chain C) with the IgA Fc are slightly different from that of the first molecule (chain. D). The SSL7 chain C (residues; 14, 18, 36-39, 55, 78-83, 87, 89) contact the IgA Fc chain B (residues; 257-258, 313, 316, 389, 433, 436-437, 439-443, 445, 447, 449, 450). The Tyr11 residue of this SSL7 molecule (C chain) does not contact the IgA Fc A chain. Some of these differences in the contacts of the two SSL7 molecules may result from the asymmetry of the IgA Fc, from differing mobility of the N-termini of the two SSL7 molecules in the crystal complex or in some instances may fall outside the 4 Å definition of a contact, but may actually be contacts given there is a working uncertainty of ±0.5 Å in the structure. Thus the full definition of contacts is described by the combined contacts of the first SSL7 (chain D) and the second SSL7 molecule (chain C) in the complex, that is SSL7 residues 11, 14, 18, 36-39, 55, 78-83, 85, 87, 89, 179.

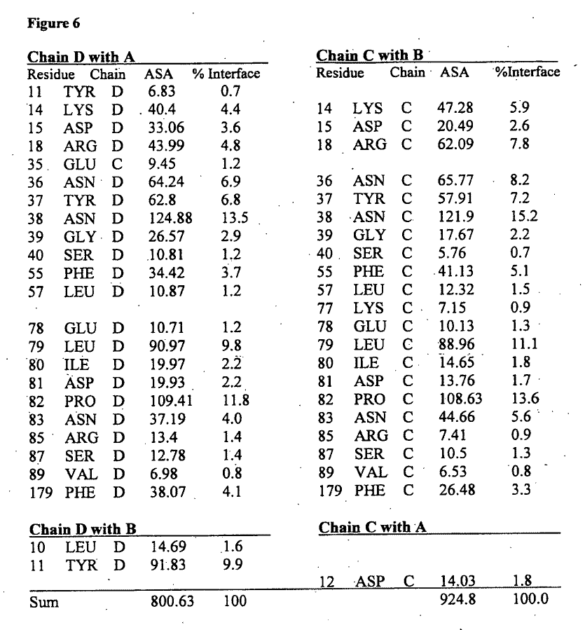

Some residues are not contacts in the interface but contribute to the buried surface area of the interface, such that mutation of these residues would be likely to affect the SSL7:IgA interaction. The buried surface of the complex was determined using the Protein-Protein Interaction Server (35). FIG. 6 provides a summary of the residues that contribute to the buried surface area defined by a 5 Å probe radius. The SSL7 D chain residues Leu10, Tyr11, Lys14, Asp15, Arg18, Asn36, Tyr37, Asn38, Gly39, Ser40, Phe55, Leu57, Glu78, Leu79, Ile80, Asp81, Pro82, Asn83, Arg85, Ser87, Val89 and Phe179 contribute to the buried surface area of the interface with the IgA Fc A chain residues Leu256, Leu257, Leu258, Gly259, Glu313, Asn316, His317, Lys340, Arg382, Leu384, Glu389, Thr429, Met433, Glu437, Leu439, Pro440, Leu441, Ala442, Phe443, Thr444, Gln445, Lys446, Thr447, Asp449 and Arg450 and the IgA Fc B chain residues Ser356, Glu357 and Ala360.

The SSL7 C chain residues Asp12, Lys14, Asp15, Arg18, Glu35, Asn36, Tyr37, Asn38, Gly39, Ser40, Phe55, Leu57, Lys77, Glu78, Leu79, Ile80, Asp81, Pro82, Asn83, Arg85, Ser87, Val89 and Phe179 contribute to the buried surface area of the interface with the IgA Fc B chain residues Leu256, Leu257, Leu258, Glu313, Asn316, His317, Arg382, Glu389, Thr429, Met433, Glu437, Leu439, Pro440, Leu441, Ala442, Phe443, Thr444, Gln445, Lys446, Thr447, Ile448, Asp449 and Arg450 and the IgA Fc A chain residues Ser356, and Glu357.

Taken together the SSL7 residues Leu10, Tyr11, Asp12, Lys14, Asp15, Arg18, Glu35, Asn36, Tyr37, Asn38, Gly39, Ser40, Phe55, Leu57, Lys77, Glu78, Leu79, Ile80, Asp81, Pro82, Asn83, Arg85, Ser87, Val89 and Phe179 contribute to the buried surface area of an interface with the IgA Fc.

### Mutants of SSL7

### Methods and Materials

### SSL7 Gene and Amino Acid Sequence

The SSL7 gene sequence used to generate SSL7 mutants was obtained from a Staphylococcus aureus isolate obtained from GreenLane hospital, Auckland New Zealand and designated strain number 4427. Using standard procedures the DNA sequence of the SSL7 gene was determined and the amino acid sequence translated. The nucleotide and amino acid sequences are provided below.

SEQ ID No: 1-
SSL7 (4427) Nucleic Acid Sequence
5′-
GTACAACATTTATATGATATTAAAGACTTACATCGATACTACTCATCAGA
AAGTTTTGAATTCAGTAATATTAGTGGTAAGGTTGAAAATTATAACGGTT
CTAACGTTGTACGCTTTAACCAAGAAAATCAAAATCACCAATTATTCTTA
TTAGGTAAAGATAAAGAGAAATATAAAGAAGGCATTGAAGGCAAAGATGT
CTTTGTGGTAAAAGAATTAATTGATCCAAACGGTAGATTATCTACTGTTG
GTGGTGTGACTAAGAAAAATAACAAATCTTCTGAAACTAATACACATTTA
TTTGTTAATAAAGTGTATGGCGGAAATTTAGATGCATCAATTGACTCATT
TTCAATTAATAAAGAAGAAGTTTCACTGAAAGAACTTGATTTCAAAATTA
GACAACATTTAGTTAAAAATTATGGTTTATATAAAGGTACGACTAAATAC
GGTAAGATCACTATCAATTTGAAAGATGGAGAAAAGCAAGAAATTGATTT
AGGTGATAAATTGCAATTCGAGCGCATGGGTGATGTGTTGAATAGTAAGG
ATATTAATAAGATTGAAGTGACTTTGAAACAAATT -3′
SEQ ID No: 2
Translated SSL7 (4427) amino acid sequence
VQHLYDIKDLHRYYSSESFEFSNISGKVENYNGSNVVRFNQEKQNHQLFL
LGEDKAKYKQGLQGQDVFVVKELIDPNGRLSTVGGVTKKNNQSSETNIHL
LVNKLDGGNLDATNDSFLINKEEVSLKELDFKIRKQLVEKYGLYQGTSKY
GKITIILNGGKKQEIDLGDKLQFERMGDVLNSKDINKIEVTLKQI

### Construction and Purification of Mutants

Mutants at individual residues of SSL7 were produced by overlap PCR as previously described (36) using synthetic oligonucleotides listed in Table 1. Generally, synthetic oligonucleotides were constructed (table 1) with a single base change that would alter the specific amino acid to be targeted. The oligonucleotides were designed to overlap by at least 8 base pairs. The oligonucleotides were used to prime separate amplification reactions with SSL7 at the DNA template in the vector pBluescript using universal oligonucleotides that were complementary to each side of the pBluescript multi-cloning site. The two DNA products resulting from the first amplification were mixed together and re-amplified with the pBluescript universal oligonucleotides to produce the full length SSL7 molecule with the desired mutation. The final PCR product was cleaved with restriction enzymes within the multicloning site, and inserted into a vector for DNA sequencing.

TABLE I
Oligonucleotide pairs used in mutagenesis

| Mutant | Primers |
|---|---|
| Y37A | U 5′-GAAAACGCCAATGGTTCTAACG |
| | (SEQ ID NO. 6) |
| | L 5′-CATTGGCGTTTTCAACCTTACC |
| | (SEQ ID NO. 7) |
| N38T | U 5′-GGTAAGGTTGAAAATTATACCGGTTC |
| | (SEQ ID NO. 8) |
| | L 5′-CAACGTTAGAACCGGTATAATTTTC |
| | (SEQ ID NO. 9) |
| R44A | U 5′-CGGTTCTAACGTTGTAGCCTTTAACC |
| | (SEQ ID NO. 10) |
| | L 5′-GATTTTCTTGGTTAAAGGCTACAACG |
| | (SEQ ID NO. 11) |
| L79A | U 5′-GTCTTTGTGGTAAAAGAAGCAATTGATCC |
| | (SEQ ID NO. 12) |
| | L 5′-CCGTTTGGATCAATTGCTTCTTTTACC |
| | (SEQ ID NO. 13) |
| P82A | U 5′-GGTAAAAGAATTAATTGATGCAAACGG |
| | (SEQ ID NO. 14) |
| | L 5′-CAGTAGATAATCTACCGTTTGCATCAAT |
| | (SEQ ID NO. 15) |
| N83A | U 5′-GGTAAAAGAATTAATTGATCCAGCCGGTAG |
| | (SEQ ID NO. 16) |
| | L 5′-CCAACAGTAGATAATCTACCGGCTGGATC |
| | (SEQ ID NO. 17) |
| R85A | U 5′-CACACCACAACAGTAGATAATGCACCGTTTGGATCAATTAATTC |
| | (SEQID NO. 18) |
| | L 5′-GAATTAATTGATCCAAACGGTGCATTATCTACTGTTGGTGGTGTG |
| | (SEQ ID NO. 19) |

Mutants containing the desired single point mutation were confirmed by DNA sequencing and cloned into the expression vector pET32 3C—a modified version of the commercially available vector pET32a (Novagen) which contains a sequence coding for a cleavage site for the viral protease 3C between the thioredoxin gene sequence and the inserted gene. Recombinant plasmid DNA was used to transform E. coli and transformants were grown in Terrific Broth. Expression was initiated with the addition of 0.1 mM IPTG and cultures continued until stationary phase was reached. Recombinant SSL7 was purified from lysed bacteria using Ni²⁺ IDA Sepharose chromatography as previously described (37). SSL7 mutants were purified and stored at 1 mg/ml in 50 mM PO₄ buffer pH 6.8.

### IgA Binding Affinity of SSL7 Mutants

Binding affinities were examined using a BIAcore biosensor as described (37). Human serum IgA was purified by passing human serum diluted 1:2 with phosphate buffered saline over a 1 ml affinity column of SSL7 Sepharose (37, or WO2005/090381). IgA was eluted from the column with 50 mM Glycine pH 3.5, neutralised with 1M Tris pH8.0. IgA was further purified to remove residual C5 protein on a Superdex 200 FPLC column and stored at 1 mg/ml in 50 mM PO₄ buffer pH6.8. Purified human IgA was used to coat a BIAcore CM5 biosensor chip using carbodiimide chemistry to ˜200 RU as previously described (37). Purified SSL7 mutants were passed across the surface of the chip over a concentration range varying from 10-200 nM at a flow rate of 30 μl/min. The binding and dissociation kinetics were globally fitted using the BIAevaluation software version 2.1. The equilibrium binding of SSL7 mutants was evaluated over 120 minute injections using a concentration range of 0.25-400 nM range and the equilibrium (R_{eq}) at 120 minutes was fitted to the two site binding model R_{eq}=B1×A/(KD1+A)+B2×A/(KD2+A) where B1, B2, KD1, and KD2 are the respective binding capacities and dissociation constants of the two sites, and A is the free analyte concentration.

### Results

### Effects of Individual Mutations on SSL7 Binding to IgA Fc.

The effect of individual mutations on the binding affinity of SSL7 to IgA was measured by Biosensor analysis and the quantitative values for each mutant are provided in Table 2. The results from mutants are consistent with their predicted position in the interface between SSL7 and IgA Fc and their calculated contributions to the interface surface (FIG. 6). The L79A mutation had the largest impact on binding, reducing the affinity as measured by the dissociation constant of binding 91-fold. L79 contributes 9.8% of the interface. From the crystal structure, the most significant contributions, to binding are made by two regions N36.Y37.N38 which contributes a total of ˜30% of the interface surface and L79.P82.N83 which contributes ˜30% of the interface. A third point of contact is identified through the residues K14.R18 which contributes ˜14% of the total surface of the interface. Mutants made in the first site include Y37A and N38T. The N38T mutation had less impact on binding perhaps because Threonine partially substituted for Asparagine. The mutation P82A reduced binding affinity by over 30-fold, consistent with its significant contribution (˜13%) to the interface. Combining mutations at Y37A.N38A and P82A.N8A into a single molecule would likely produce an SSL7 that has substantially reduced binding to IgA Fc.

Each SSL7 mutant protein was tested for its ability to inhibit the activity of human complement in a standardised complement haemolytic assay. In this assay, fresh human serum from one individual containing active complement previously found to haemolyse red blood cells from another individual, was first mixed with varying concentrations of SSL7 mutant to allow SSL7 and complement to bind then incubated for 1 hour at 37° C. with purified red blood cells. The degree of complement mediated haemolysis was measured by absorbance at 412 nm as described in detail (37). The inhibition of each mutant was measured against the inhibition obtained by wild-type SSL7 protein. All mutants that showed reduced binding to IgA (presented in table 2) showed no loss of inhibitory activity on complement mediated haemolysis.

TABLE 2
Comparative dissociation constants (K_{D}) of SSL (GL1 allele)
mutants in the observed binding site to human serum IgA.

| SSL7 mutant | K_{D} × 10⁻⁶ M | Chi squared | Change |
|---|---|---|---|
| SSL7 wild-type* | 0.0011 | 0.184 | 0 |
| N38T | 0.038 | 2.74 | 35 |
| R44A | 0.0036 | 21.9 | 3.2 |
| L79A | 0.1 | 1.87 | 91 |
| P82A | 0.039 | 1.75 | 35 |
| N83A | 0.004 | 10.6 | 4 |

*From reference 37

### C-Terminal Fragment of SSL7

### Materials and Methods

### SSL7 Gene and Amino Acid Sequence

SSL7 sequence from the Staphylococcus aureus strain 4427 was used to generate the C-terminal fragment. The nucleic acid sequence of the C-terminal fragment of SSL7 4427 is:

AGCAGCGAAACCAACACCCATCTGTTTGTGAACAAAGTGTATGGCGGCAA
CCTGGATGCGAGCATTGATAGCTTTAGCATTAACAAAGAAGAAGTGAGCC
TGAAAGAACTGGATTTTAAAATTCGCCAGCATCTGGTGAAAAACTATGGC
CTGTATAAAGGCACCACCAAATATGGCAAAATTACCATTAACCTGAAAGA
TGGCGAAAAACAGGAAATTGATCTGGGCGATAAACTGCAGTTTGAACGCA
TGGGCGATGTGCTGAACAGCAAAGATATTAACAAAATTGAAGTGACCCTG
AAACAGATT.

This sequence is designated herein as SEQ ID No. 20.

### Construction and Purification of C-Terminal Fragment

The C-Terminal fragment was generated as per the SSL7 protein as described by Langley et al (31). Forward primer used was F 5′ CGC GGA TCC TCT GAA ACT AAT ACA C (SEQ ID No. 32).

The sequence of the SSL7 C-terminus fragment is SSETNTHLFVNKVYGGNLDASIDSFSINKEEVSLKELDFKIRQHLVKNYGLYKGTT KYGKITINLKDGEKQEIDLGDKLQFERMGDVLNSKDINKIEVTLKQI (SEQ ID No. 3), spanning from position 99 to 201 in the native SSL7 protein from strain 4427.

### Blood and Serum

Human blood was collected into EDTA covered tubes and keep on ice. Serum was gained by centrifugation for 25 mins at 1700 rpm at 4° C. Supernatant serum were taken and pooled and stored on ice. Inhibitor solution was added in a ration 1:20 (1 part Inhibitor Solution to 20 parts serum). Inhibitor Solution: 1M KH2PO4, 0.2M Na2 EDTA, 0.2M Benzamidine. 0.1M PMSF in anhydrous isopropyl alcohol was made up fresh and added to the serum-Inhibitor Solution to a final concentration of 1 mM. Inhibitor treated serum was passed over lysine sepharose column immediately at 4° C.

### Lysine Sepharose Column

Lysine Sepharose is made by coupling lysine hydrochloride to CNBr activated Sepharose (Sigma). 50 mM lysine hydrochloride in phosphate buffered saline pH8.3 is incubated overnight at 4° C. with CNBr activated Sepharose and then the matrix is washed with water and incubated for a further 24 hrs at 4° C. in 0.1M Tris pH8.0 to deactivate residual sites. The lysine Sepharose is washed with 5 L of water and stored in 20% ethanol for future use.

Lysine Sepharose was used as an affinity matrix to remove serum plasminogen prior to purification of complement C5. 100 ml of freshly obtained human serum was passed over a 50 ml column of lysine Sepharose and the passthrough fractions collected into tubes containing enough 1M EACA to provide a final concentration of 0.2M Epsilon Amino Caproic Acid (EACA) on ice. Serum depleted of plasminogen by passage over lysine Sepharose was immediately passed over the SSL7 C-terminal column at 4° C.

### SSL7 C-Terminal Fragment Column

CNBr-activated sepharose 4B beads were coated with purified SSL7 C-terminus protein. Serum was passed over the column and the column washed and C5 eluted. Washing and elution steps were performed with 3 fractions of 3 ml of liquid. The second fraction was incubated on the column for 5-10 minutes to assure effectiveness (if not mentioned differently). Wash and elution were collected into tubes containing the following (given in final concentrations): 0.1M tris pH 8.0 (except 0.25M glycine pH 2.95 elution which was collected into 0.1M tris base), 0.1M EACA, 0.01M EDTA, 0.001M PMSF, and 0.01M Benzamidine.

### Washing of SSL7 C-Terminal Column

After inhibitor treated serum was passed over the column and column was washed with 10 ml Washing Buffer (100 mM Na phosphate pH 7.4, 20 mM EDTA, 300 mM NaCl), 1M MgCl₂, 0.1M glycine pH 4.5, 0.1M glycine pH 4.0, 0.1M glycine pH 3.5. All glycine solutions were made up in MilliQ and pH was adapted with concentrated HCl and sterile filtered.

### Elution of SSL7 C-Terminal Column

C5 elution of the column was performed using 0.1M glycine pH 3.0 and 0.25M glycine pH 2.95. Dialysis was performed over night at 4° C. into 10 mM phosphate pH 7.2 and 150 mM NaCl. Dialysed C5 was sterile filtered prior to concentration. Concentration was performed by centrifugation using Vivaspin20 10000 MWCO PES. C5 was rapidly frozen in a dry ice ethanol bath and then stored at −80° C.

### Results

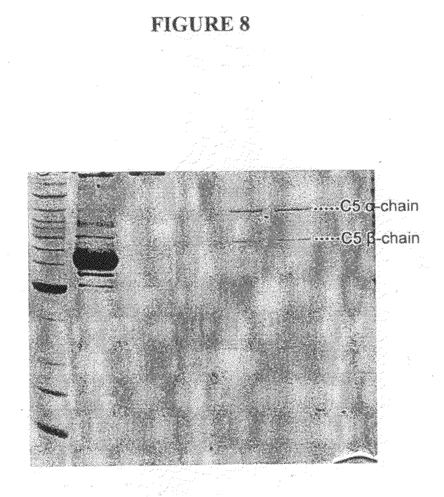

FIG. 8 illustrates the results obtained from running serum over an SSL7 C-terminal fragment column. The yield of C5 was 0.5 mg from 20 ml of serum.

Amino Acids in the C-Terminal Domain which Affect C5 Binding

### Materials and Methods

### Mutants

Mutants were generated using the techniques described herein before and the synthetic oligonucleotide primers in Table 3 below.

TABLE 3
Oligonucleotide pairs used in mutagenesis.
Point Mutations are highlighted in bold
letters.

| Mutant | Primers |
|---|---|
| D117A | U 5′- GTG TAT GGC GGA AAT TTA GCT GCA |
| | TCA ATT GAC TC |
| | (SEQ ID No. 28) |
| | L 5′- GA GTC AAT TGA TGC AGC TAA ATT |
| | TCC G |
| | (SEQ ID No. 29) |
| E170A | F 5′ GAT GGA GAA AAG CAA GCA ATT GAT |
| | TTA GG |
| | (SEQ ID No. 30) |
| | R 5′ C ACC TAA ATC AAT TGC TTG CTT TTC |
| | TCC |
| | (SEQ ID No. 31) |

### Preparation of Human Red Blood Cells (RBC)

5 ml of red blood cells (RBC) were added to 45 ml of GVB containing 10 mM MgCl₂ and 1 mM ethylene glycol tetraacetic acid (from now on referred to as EGTA) and incubated for 15 min at 37° C. Cells were centrifuged at 1250×g for 5-10 minutes at 4° C. Supernatant was removed and the cells were resuspended in ice cold buffer (GVB containing 10 mM MgCl₂ and 1 mMEGTA, stored in −20° C.). Procedure was repeated until supernatant was clear following centrifugation. Cells were standardized to 2×10⁸ cells/ml

### Complement Mediated Haemolytic Assay

Human Serum was diluted 2 fold with GVB containing 10 mM MgCl₂ and 1 mM EGTA. In a 96 well plate samples were mixed as followed: 2 molar Protein (different SSL7 C-terminus mutants) in 100 ul diluted serum were added to 10⁷ RBC. 2 fold dilution into GVB (10 mM MgCl₂/1 mM EGTA) towards the next row was performed. The plate was incubated for 1 hour at 37° C. without shaking. After the incubation time cells were pelleted by centrifuging at 1250×g for 5 min. Afterwards the reaction was stopped by adding the supernatant into ice cold 0.15M NaCl in a ratio 1:1.5. Absorbance was read at A₄₁₂ₙₘ in a spectrometer.

This assay was performed to test the ability of the mutant SSL7 C-terminus fragment to bind C5. The ability of C-terminus to bind C5 prevents the activation of C5 and therefore heamolysis of the red blood cells. Higher levels of haemolysis therefore represent a SSL7 C-terminal mutant that has lost some ability to bind C5.

### Results

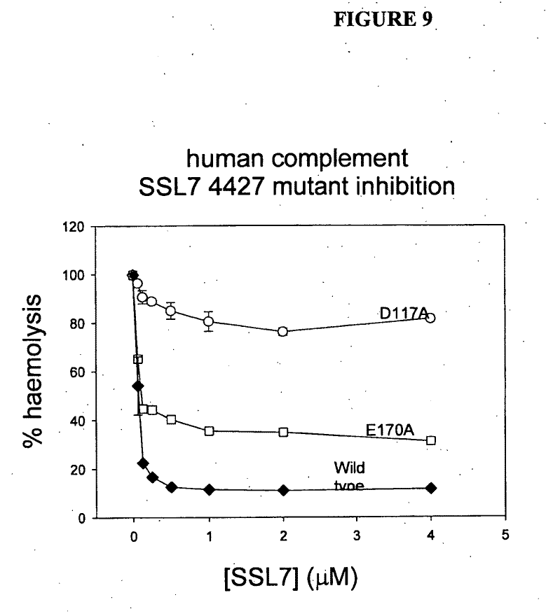

The results of this experiment are illustrated in FIG. 9. SSL7 wild-type completely inhibited complement mediate lysis above 150 nM. At 2 μM SSL7 wild-type, haemolysis was only 10% of the maximum lysis or 90% inhibition. At 2 μM, mutant E170A lysis was 35% of the total or 65% inhibition. At 2 μM, mutant D117A lysis was 80% which equates to 20% inhibition. The results indicate the importance of the amino acids at this position to C5 binding.

The invention has been described herein with reference to certain preferred embodiments, in order to enable the reader to practice the invention without undue experimentation. Those skilled in the art will appreciate that the invention is susceptible to variations and modifications other than those specifically described. It is to be understood that the invention includes all such variations and modifications. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

The reference to any prior art in this specification is not, and should not be taken as, an acknowledgment, or any form of suggestion, that that prior art forms part of the common general knowledge in the field of endeavour to which the invention relates in any country.

Throughout this specification, and any claims which follow, unless the context requires otherwise, the words “comprise”, “comprising” and the like, are to be construed in an inclusive sense as opposed to an exclusive sense, that is to say, in the sense of “including, but not limited to”.

### REFERENCES

1. Van Belkum, A., M. Kools-Sijmons, and H. Verbrugh. 2002. Attachment of Staphylococcus aureus to eukaryotic cells and experimental pitfalls in staphylococcal adherence assays: a critical appraisal. J Microbiol Methods 48:19-42.
2. Novick, R. P., P. Schlievert, and A. Ruzin. 2001. Pathogenicity and resistance islands of staphylococci. [Review][57 refs]. Microbes & Infection 3:585-594.
3. Kuroda, M., T. Ohta, I. Uchiyama, T. Baba, H. Yuzawa, I. Kobayashi, L. Cui, A. Oguchi, K. Aoki, Y. Nagai, J. Lim., T. Ito, M. Kanamori, H. Matsumaru, A. Maruyama, H. Murakami, A. Hosoyama, Y. Mizutani-Ui, N. K. Takahashi, T. Sawano, R. Inoue, C. Kaito, K. Sekimizu, H. Hirakawa, S. Kuhara, S. Goto, J. Yabuzaki, M. Kanehisa, A. Yamashita, K. Oshima, K. Furuya, C. Yoshino, T. Shiba, M. Hattori, N. Ogasawara, H. Hayashi, and K. Hiramatsu. 2001. Whole genome sequencing of meticillin-resistant Staphylococcus aureus. Lancet. 357:1225-1240.
4. Novick, R. P. 2003. Mobile genetic elements and bacterial toxinoses: the superantigen-encoding pathogenicity islands of Staphylococcus aureus. Plasmid 49:93-105.
5. Fraser, J., V. Arcus, P. Kong, E. Baker, and T. Proft. 2000. Superantigens—powerful modifiers of the immune system. Mol Med Today 6:125-132.
6. Arcus, V. L., T. Proft, J. A. Sigrell, H. M. Baker, J. D. Fraser, and E. N. Baker. 2000. Conservation and variation in superantigen structure and activity highlighted by the three-dimensional structures of two new superantigens from Streptococcus pyogenes. Journal of Molecular Biology 299:157-168.
7. Arcus, V. 2002. OB-fold domains: a snapshot of the evolution of sequence, structure and function. Current Opinion in Structural Biology 12:794-801.
8. Williams, R. J., J. M. Ward, B. Henderson, S. Poole, B. P. O'Hara, M. Wilson, and S. P. Nair. 2000. Identification of a novel gene cluster encoding staphylococcal exotoxin-like proteins: characterization of the prototypic gene and its protein product, SSL7. Infection & Immunity. 68:4407-4415.
9. Kim, J., R. Urban, J. L. Strominger, and D. Wiley. 1994. Toxic Shock Syndrome Toxin-1 Complexed with a Major Histocompatibility Molecule HLA-DR1. Science 266:1870-1874.
10. Baba, T., F. Takeuchi, M. Kuroda, H. Yuzawa, K. Aoki, A. Oguchi, Y. Nagai, N. Iwama, K. Asano, T. Naimi, H. Kuroda, L. Cui, K. Yamamoto, and K. Hiramatsu. 2002. Genome and virulence determinants of high virulence community-acquired MRSA. Lancet 359:1819-1827.
11. Arcus, V. L., R. Langley, T. Proft, J. D. Fraser, and E. N. Baker. 2002. The Three-dimensional Structure of a Superantigen-like Protein, SSL3, from a Pathogenicity Island of the Staphylococcus aureus Genome. J Biol Chem 277:32274-32281.
12. Fitzgerald, J. R., D. E. Sturdevant, S. M. Mackie, S. R. Gill, and J. M. Musser. 2001. Evolutionary genomics of Staphylococcus aureus: insights into the origin of methicillin-resistant strains and the toxic shock syndrome epidemic. Proc Natl Acad Sci USA 98:8821-8826.
13. Kerr, M. A. 1990. The structure and function of human IgA. Biochemical Journal. 271:285-296.
14. Van Spriel, A. B., J. H. Leusen, H. Vile, and J. G. Van De Winkel. 2002. Mac-1 (CD11b/CD18) as accessory molecule for Fc alpha R (CD89) binding of IgA. J Immunol 169:3831-3836.
15. van Egmond, M., C. A. Damen, A. B. van Spriel, G. Vidarsson, E. van Garderen, and J. G. J. van de Winkel. 2001. IgA and the IgA Fc receptor. Trends in Immunology 22:205-211.
16. Corthesy, B. 2002. Recombinant immunoglobulin A: powerful tools for fundamental and applied research. Trends in Biotechnology 20:65-71.
17. Herr, A. B., E. R. Ballister, and P. J. Bjorkman. 2003. Insights into IgA-mediated immune responses from the crystal structures of human FcaII and its complex with IgA1-Fc. Nature 423:614-620.
18. Morton, H. C., and P. Brandtzaeg. 2001. CD89: the human myeloid IgA Fc receptor. Archivum Immunologiae et Therapiae Experimentalis. 49:217-229.
19. van Egmond, M., E. van Garderen, A. B. van Spriel, C. A. Damen, E. S. van Amersfoort, G. van Zandbergen, J. van Hattum, J. Kuiper, and J. G. van de Winkel. 2000. FcalphaRI-positive liver Kupffer cells: reappraisal of the function of immunoglobulin A in immunity. Nature Medicine. 6:680-685.
20. Pleass, R., J. Dunlop, C. Anderson, and J. Woof. 1999. Identification of residues in the CH2/CH3 domain interface of IgA essential for interaction with the human fcalpha receptor (FcalphaR) CD89. Journal of Biological Chemistry 274:23508-23514.
21. Carayannopoulos, L., J. Hexham, and J. Capra. 1996. Localization of the binding site for the monocyte immunoglobulin (Ig) A-Fc receptor (CD89) to the domain boundary between Calpha2 and Calpha3 in human IgA1. Journal of Experimental Medicine.
22. Wines, B., M. Hulett, G. Jamieson, H. Trist, J. Spratt, and P. Hogarth. 1999. Identification of residues in the first domain of human Fc alpha receptor essential for interaction with IgA. Journal of Immunology 162:2146-2153.
23. Nilsson, U. R., R. J. Mandle, Jr, and J. A. McConnell-Mapes. 1975. Human C3 and C5: subunit structure and modifications by trypsin and C42-C423. J Immunol 114:815-822.
24. Tack, B. F., S. C. Morris, and J. W. Prahl. 1979. Fifth component of human complement: purification from plasma and polypeptide chain structure. Biochemistry 18:1490-1497.
25. Gerard, N. P., and C. Gerard. 1991. The chemotactic receptor for human C5a anaphylatoxin. Nature 349:614-617.
26. Wines, B., C. Sardjono, H. Trist, C. Lay, and P. Hogarth. 2001. The Interaction of FcalphaRI with IgA and Its Implications for Ligand Binding by Immunoreceptors of the Leukocyte Receptor Cluster. Journal of Immunology: 1781-1789.
27. Shevach, E. N. 1994. Complement. In Current Protocols in Immunology. W. Strober, editor. John Wiley and Sons Ltd, New York. 13.10.11.
28. Morton, H. C., M. van Egmond, and J. G. van de Winkel. 1996. Structure and function of human IgA Fc receptors (Fc alpha R). Critical Reviews in Immunology. 16:423-440.
29. Shevchenko, A., M. Wilm, O. Vorm, and M. Mann. 1996. Mass spectrometric sequencing of proteins silver-stained polyacrylamide gels. Anal Chem 68:850.
30. Evans, M. J., S. L. Hartman, D. W. Wolff, S. A. Rollins, and S. P. Squinto. 1995. Rapid expression of an anti-human C5 chimeric Fab utilizing a vector that replicates in COS and 293 cells. J Immunol Methods 184:123-138.
31. Langley, R., B. Wines, N. Willoughby, I. Basu, T. Proft, and J. D. Fraser. 2005. The staphylococcal superantigen-like protein 7 binds IgA and complement C5 and inhibits IgA-FcalphaRI binding and serum killing of bacteria. J Immunol 174:2926-2933.
32. Wines, B. D., N. Willoughby, J. D. Fraser, and P. M. Hogarth. 2006. A competitive mechanism for staphylococcal toxin SSL7 inhibiting the leukocyte IgA receptor, Fc alphaRI, is revealed by SSL7 binding at the C alpha2/C alpha3 interface of IgA. J Biol Chem 281:1389-1393.
33. Diemand, A. V., and H. Scheib. 2004. iMolTalk: an interactive, internet-based protein structure analysis server. Nucleic Acids Res 32:W512-516.
34. Diemand, A. V., and H. Scheib. 2004. MolTalk—a programming library for protein structures and structure analysis. BMC Bioinformatics 5:39.
35. Jones, S., and J. M. Thornton. 1996. Principles of protein-protein interactions. Proc Natl Acad Sci USA 93:13-20.
36. Hudson K R, Robinson H, Fraser J D. 1993. Two adjacent residues in staphylococcal enterotoxins A and E determine T cell receptor V beta specificity. J Exp Med 177: 175-84
37. Langley R, Wines B, Willoughby N, Basu I, Proft T, Fraser J D. 2005. The staphylococcal superantigen-like protein 7 binds IgA and complement C5 and inhibits IgA-Fc alpha RI binding and serum killing of bacteria. J Immunol 174: 2926-33

1. An SSL7 mutant having the ability to bind C5 but no or reduced ability to bind IgA.

2. An SSL7 mutant as claimed in claim 1 wherein the mutant comprises an SSL7, allelic variant or functional equivalent thereof including one or more mutation in the IgA binding region.

3. An SSL7 mutant as claimed in claim 2 wherein the mutant comprises an SSL7, allelic variant or functional equivalent thereof including a mutation at one or more of the following amino acid sites: 11, 14, 18, 36, 37, 38, 39, 55, 78, 79, 80, 81, 82, 83, 85, 87, 89 and 179.

4. An SSL7 mutant as claimed in claim 3 wherein the mutant comprises an SSL7, allelic variant or functional equivalent thereof including a mutation at one or more of the following amino acid sites: Tyr11, Lys14, Arg18, Asn36, Tyr37, Asn38, Gly39, Phe55, Glu78, Leu79, Ile80, Asp81, Pro82, Asn83, Arg85, Ser87, Val89 and Phe179.

5. An SSL7 mutant as claimed in claim 2 wherein the mutant comprises an SSL7, allelic variant or functional equivalent thereof including a mutation at one or more of the following amino acid sites: Leu10, Tyr11, Asp12, Lys14, Asp15, Arg18, Glu35, Asn36, Tyr37, Asn38, Gly39, Ser40, Phe55, Leu57, Lys77, Glu78, Leu79, Ile80, Asp81, Pro82, Asn83, Arg85, Ser87, Val89 and Phe179.

6. An SSL7 mutant as claimed in claim 2 wherein the mutant comprises an SSL7, allelic variant or functional equivalent thereof including a mutation at one or more of the following amino acid sites: Glu35, Ser40, Asn41, Val42, Arg44, Gln50, Asn 51, His52, Gln53, Leu54, Leu56, Leu57, Lys61, Val76, Lys77, Gly84, Leu86, Ser87, Thr88, Gly90, Lys133, Lys176, and Met182.

7. An SSL7 mutant as claimed in claim 2 wherein the mutant is chosen from an SSL7, allelic variant or functional equivalent thereof including a mutation at one or more of the following amino acid sites: 37, 38, 44, 79, 81, 82, 83, and 85.

8. An SSL7 mutant as claimed in claim 7 wherein the mutant is chosen from an SSL7, allelic variant or functional equivalent thereof including one or more of the following mutations: Y37A, N38T, R44A, L79A, D81A, P82A, N83A, and R85A.

9. An SSL7 mutant as claimed in claim 1 wherein the IgA binding region is deleted.

10. An SSL7 mutant as claimed in claim 9 wherein the mutant comprises a C-terminal fragment of SSL7.

11. An SSL7 mutant as claimed in claim 10 wherein the mutant comprises the amino acid sequence:
SSETNTHLFVNKVYGGNLDASIDSFSINKEEVSLKELDFKIRQHLVKNYG
LYKGTTKYGKITINLKDGEKQEIDLGDKLQFERMGDVLNSKDINKIEVTL
KQI.

12. An isolated nucleic acid encoding an SSL7 mutant as claimed in claim 1.

13. A method of isolating C5 present in a sample, the method comprising at least the steps of:
a) Bringing an SSL7 mutant having the ability to bind C5 but no or reduced ability to bind IgA in contact with the sample for a period sufficient to allow the SSL7 mutant to bind to C5 to form a complex;
b) Separating the complex; and
c) Releasing C5 from the complex.

14. A method for isolating C5 from a sample, the method comprising at least the steps of:
a) Providing a matrix to which an SSL7 mutant having the ability to bind C5 but no or reduced ability to bind IgA is bound;
b) Providing a sample;
c) Bringing said matrix and said sample into contact for a period sufficient to allow the SSL7 mutant to bind to C5 present in the sample; and,
d) Releasing C5 from the matrix.

15. A method as claimed in claim 13 wherein the method further comprises the step of collecting the C5 released.

16. A method as claimed in claim 14 wherein the matrix is in the form of a column over which the sample is passed.

17. A method as claimed in claim 14 wherein the method further comprises the step of washing contaminants present in the sample from the matrix prior to release of C5.

18. A method as claimed in claim 14 wherein the matrix is Sepharose.

19. A method as claimed in claim 13 wherein the sample is milk, colostrum, or serum.

20. A method as claimed in claim 13 wherein the method further comprises the step of determining the quantity of C5 present in the sample.

21. A method as claimed in claim 13 wherein C5 is released using a low pH buffer such as 50 mM acetate pH 3.5.

22. A method of detecting C5 in a sample, the method comprising at least the steps of:
a) Contacting a sample with an SSL7 mutant having the ability to bind C5 but no or reduced ability to bind IgA for a period sufficient to allow the SSL7 mutant to bind to C5; and,
b) Detecting bound SSL7.

23. A method as claimed in claim 22 wherein the method further includes the step of determining or quantifying the level of bound SSL7.

24. A method as claimed in claim 22 wherein the method is conducted for the purpose of diagnosing C5 abnormalities in a subject.

25. A method as claimed in claim 22 wherein the subject is a mammal, more preferably a human.

26. A method of removing C5 from a sample, the method comprising at least the steps of:
a) Bringing an SSL7 mutant having the ability to bind C5 but no or reduced ability to bind IgA in contact with the sample for a period sufficient to allow the SSL7 mutant to bind to C5 to form a complex;
b) Separating the complex from the sample.

27. A kit for the detection, isolation, or removal of C5 in a sample, the kit comprising at least an SSL7 mutant having the ability to bind C5 but no or reduced ability to bind IgA.

28.-29. (canceled)


SEQUENCE LISTING (36 sequences)

SEQ ID NO: 1 - DNA, 585 bp, Staphylococcus aureus
gtacaacatttatatgatattaaagacttacatcgatactactcatcagaaagttttgaattcagtaatattagtggtaaggttgaaaattataacggttctaacgttgtacgctttaaccaagaaaatcaaaatcaccaattattcttattaggtaaagataaagagaaatataaagaaggcattgaaggcaaagatgtctttgtggtaaaagaattaattgatccaaacggtagattatctactgttggtggtgtgactaagaaaaataacaaatcttctgaaactaatacacatttatttgttaataaagtgtatggcggaaatttagatgcatcaattgactcattttcaattaataaagaagaagtttcactgaaagaacttgatttcaaaattagacaacatttagttaaaaattatggtttatataaaggtacgactaaatacggtaagatcactatcaatttgaaagatggagaaaagcaagaaattgatttaggtgataaattgcaattcgagcgcatgggtgatgtgttgaatagtaaggatattaataagattgaagtgactttgaaacaaatt

SEQ ID NO: 2 - protein, 135 aa, Stapylococcus areus
VQHLYDIKDLHRYYSSESFEFSNISGKVENYNGSNVVRFNQEKQNHQLFLLGEDKAKYKQGLQGQDVFVVKELIDPNGRLSTVGGVTKKNNQSSETNIHLLVNKLDGGNLDATNDSFLINKEEVSLKELDFKIRK

SEQ ID NO: 3 - protein, 103 aa, Staphylococcus aureus
SSETNTHLFVNKVYGGNLDASIDSFSINKEEVSLKELDFKIRQHLVKNYGLYKGTTKYGKITINLKDGEKQEIDLGDKLQFERMGDVLNSKDINKIEVTLKQI

SEQ ID NO: 4 - DNA, 22 bp, Artificial Sequence
  synthetic primer
tcctgccacccccgactgtcac

SEQ ID NO: 5 - DNA, 22 bp, Artificial Sequence
  synthetic primer
ctctgacaggatacccggaagg

SEQ ID NO: 6 - DNA, 22 bp, Artificial Sequence
  synthetic primer
gaaaacgccaatggttctaacg

SEQ ID NO: 7 - DNA, 22 bp, Artificial Sequence
  synthetic primer
cattggcgttttcaaccttacc

SEQ ID NO: 8 - DNA, 26 bp, Artificial Sequence
  synthetic primer
ggtaaggttgaaaattataccggttc

SEQ ID NO: 9 - DNA, 25 bp, Artificial Sequence
  synthetic primer
caacgttagaaccggtataattttc

SEQ ID NO: 10 - DNA, 26 bp, Artificial Sequence
  synthetic primer
cggttctaacgttgtagcctttaacc

SEQ ID NO: 11 - DNA, 26 bp, Artificial Sequence
  synthetic primer
gattttcttggttaaaggctacaacg

SEQ ID NO: 12 - DNA, 29 bp, Artificial Sequence
  synthetic primer
gtctttgtggtaaaagaagcaattgatcc

SEQ ID NO: 13 - DNA, 27 bp, Artificial Sequence
  synthetic primer
ccgtttggatcaattgcttcttttacc

SEQ ID NO: 14 - DNA, 27 bp, Artificial Sequence
  synthetic primer
ggtaaaagaattaattgatgcaaacgg

SEQ ID NO: 15 - DNA, 28 bp, Artificial Sequence
  synthetic primer
cagtagataatctaccgtttgcatcaat

SEQ ID NO: 16 - DNA, 30 bp, Artificial Sequence
  synthetic primer
ggtaaaagaattaattgatccagccggtag

SEQ ID NO: 17 - DNA, 29 bp, Artificial Sequence
  synthetic primer
ccaacagtagataatctaccggctggatc

SEQ ID NO: 18 - DNA, 44 bp, Artificial Sequence
  synthetic primer
cacaccacaacagtagataatgcaccgtttggatcaattaattc

SEQ ID NO: 19 - DNA, 45 bp, Artificial Sequence
  synthetic primer
gaattaattgatccaaacggtgcattatctactgttggtggtgtg

SEQ ID NO: 20 - DNA, 309 bp, Staphylococcus aureus
agcagcgaaaccaacacccatctgtttgtgaacaaagtgtatggcggcaacctggatgcgagcattgatagctttagcattaacaaagaagaagtgagcctgaaagaactggattttaaaattcgccagcatctggtgaaaaactatggcctgtataaaggcaccaccaaatatggcaaaattaccattaacctgaaagatggcgaaaaacaggaaattgatctgggcgataaactgcagtttgaacgcatgggcgatgtgctgaacagcaaagatattaacaaaattgaagtgaccctgaaacagatt

SEQ ID NO: 21 - protein, 201 aa, Staphylococcus aureus
KEKQERVQHLYDIKDLHRYYSSESFDFSNISGKVENYNGSNVVRFNQDGQNHQLFLLGEDKAKYKQGLEGQNVFVVKELIDPNGRLSTVGGVTKKNNQSSETNTPLFVKKVYGGNLDASIESFSINKEEVSLKELDFKIRQHLVKNYGLYKGTTKYGKITFNLKDGEKKEIDLGDKLQFEHMGDVLNSKDIQNIAVTLKQI

SEQ ID NO: 22 - protein, 201 aa, Staphylococcus aureus
KEKQERVQHLYDIKDLHRYYSSESFEFSNISGKVENYNGSNVVRFNQENQNHQLFLSGKDKDKYKEGLEGQNVFVVKELIDPNGRLSTVGGVTKKNNQSSETNTPLFIKKVYGGNLDASIESFLINKEEVSLKELDFKIRQHLVKNYGLYKGTTKYGKITFNLKDGEKQEIDLGDKLQFEHMGDVLNSKDIQNIAVTINQI

SEQ ID NO: 23 - protein, 201 aa, Staphylococcus aureus
KEKQERVQHLYDIKDLHRYYSSESFEFSNISGKVENYNGSNVVRFNQEKQNHQLFLLGEDKAKYKQGLQGQDVFVVKELIDPNGRLSTVGGVTKKNNQSSETNIHLLVNKLDGGNLDATNDSFLINKEEVSLKELDFKIRKQLVEKYGLYQGTSKYGKITIILNGGKKQEIDLGDKLQFERMGDVLNSKDINKIEVTLKQI

SEQ ID NO: 24 - protein, 201 aa, Staphylococcus aureus
KEKQERVQHLYDIKDLYRYYSSESFEFSNISGKVENYNGSNVVRFNQEKQNHQLFLLGKDKDKYKKGLEGQNVFVVKELIDPNGRLSTVGGVTKKNNKSSETNTHLFVNKVYGGNLDASIDSFLINKEEVSLKELDFKIRKQLVEKYGLYKGTTKYGKITINLKDEKKEVIDLGDKLQFERMGDVLNSKDIQNIAVTINQI

SEQ ID NO: 25 - protein, 201 aa, Staphylococcus aureus; identical to SEQ ID NO: 24

SEQ ID NO: 26 - protein, 201 aa, Staphylococcus aureus
AEKQERVQHLHDIRDLHRYYSSESFEYSNVSGKVENYNGSNVVRFNPKDQNHQLFLLGKDKEQYKEGLQGQNVFVVQELIDPNGRLSTVGGVTKKNNKTSETNTPLFVNKVNGEDLDASIDSFLIQKEEISLKELDFKIRQQLVNNYGLYKGTSKYGKIIINLKDENKVEIDLGDKLQFERMGDVLNSKDIRGISVTINQI

SEQ ID NO: 27 - protein, 201 aa, Artificial Sequence
  Consensus amino acid sequence of the naturally occurring SSL7 alleles from Staphylococcus aureus
KEKQERVQHLYDIKDLHRYYSSESFEFSNISGKVENYNGSNVVRFNQEXQNHQLFLLGKDKDKYKXGLEGQNVFVVKELIDPNGRLSTVGGVTKKNNKSSETNTHLFVNKVYGGNLDASIDSFLINKEEVSLKELDFKIRQQLVXNYGLYKGTTKYGKITINLKDGXKXEIDLGDKLQFERMGDVLNSKDIQNIAVTINQI

SEQ ID NO: 28 - DNA, 35 bp, Artificial Sequence
  synthetic primer
gtgtatggcggaaatttagctgcatcaattgactc

SEQ ID NO: 29 - DNA, 27 bp, Artificial Sequence
  synthetic primer
gagtcaattgatgcagctaaatttccg

SEQ ID NO: 30 - DNA, 29 bp, Artificial Sequence
  synthetic primer
gatggagaaaagcaagcaattgatttagg

SEQ ID NO: 31 - DNA, 28 bp, Artificial Sequence
  synthetic primer
cacctaaatcaattgcttgcttttctcc

SEQ ID NO: 32 - DNA, 25 bp, Artificial Sequence
  synthetic primer s
cgcggatcctctgaaactaatacac

SEQ ID NO: 33 - protein, 211 aa, Homo sapiens
ESCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDR

SEQ ID NO: 34 - protein, 211 aa, Homo sapiens; identical to SEQ ID NO: 33

SEQ ID NO: 35 - protein, 192 aa, Staphylococcus aureus
LYDIKDLHRYYSSESFEFSNISGKVENYNGSNVVRFNQEKQNHQLFLLGEDKAKYKQGLQGQDVFVVKELIDPNGRLSTVGGVTKKNNQSSETNIHLLVNKLDGGNLDATNDSFLINKEEVSLKELDFKIRKQLVEKYGLYQGTSKYGKITIILNGGKKQEIDLGDKLQFERMGDVLNSKDINKIEVTLKQI

SEQ ID NO: 36 - protein, 192 aa, Staphylococcus aureus; identical to SEQ ID NO: 35